(19) United States
(12) Patent Application Publication
(10) Pub. No.: US 2023/0293079 A1
(43) Pub. Date: Sep. 21, 2023

(54) ELECTROCARDIOGRAM IMAGE PROCESSING METHOD AND DEVICE, MEDIUM, AND ELECTROCARDIOGRAPH

(71) Applicant: SHANGHAI MING ENTROPY PHARMACEUTICAL TECHNOLOGY CO., LTD, Shanghai (CN)

(72) Inventors: Kang Chen, Shanghai (CN); Qing Cao, Shanghai (CN)

(21) Appl. No.: 17/921,929

(22) PCT Filed: Apr. 27, 2021
(86) PCT No.: PCT/CN2021/090113

§ 371 Date: Oct. 27, 2022

(30) Foreign Application Priority Data
Apr. 30, 2020 (CN) 202010361140.7

(51) Int. Cl. A61B 5/318; A61B 5/00; G06F 18/2415

(52) U.S. Cl. CPC A61B 5/318; A61B 5/7264; A61B 5/7282; A61B 5/743; G06F 18/2415

(57) ABSTRACT

The present application relates to a field of image processing and discloses an ECG image processing method, device, medium, and an electrocardiograph. The ECG image processing method of the present application includes: receiving the ECG image; extracting a feature map of the ECG image, and reducing the feature map to obtain an attention map; extracting a feature matrix from the feature map and the attention map using a bilinear attention pooling; obtaining an expression matrix using an adaptive weight learning and a weighted fusion of the feature matrix by a multi-headed self-attention processing; and classifying the ECG image with a plurality of labels. This application allows direct interpretation of ECG images without limiting to a use of conventional digital signals, while capturing subtle discrepancies in the ECG image for classifying ECG abnormalities with high noise immunity.

### TECHNICAL FIELD

The present application relates to a field of image processing, in particular to an electrocardiogram image processing method, device, medium, and an electrocardiograph.

### BACKGROUND

Electrocardiogram (ECG) is used to reflect an electrical excitation process of heart and is an important clinical tool for cardiac examination and diagnosis. ECG generally includes static ECG, dynamic ECG and exercise ECG. In the prior art, ECG signal is learned and processed by an artificial intelligence framework of deep learning to reason about various abnormality types. ECG signal mainly serves a heart patch product to analyze common arrhythmia abnormalities based on a single-lead ECG of the heart patch product. Thus, the following deficiencies exist in the prior art. On one hand, signal processing is based on digital signal input, and because of limitations of ECG devices and storage settings of hospital information systems, a large number of ECG images are stored in clinical system in picture format. A signal processing based on digital signals limits the application of ECG signals in in practical scenarios. Most of the application are applied to cardiac patch devices, thus exclusive cooperation with the device manufacturer is required to obtain the digital signals of this device directly, making the scalability low. On the other hand, most of the prior art is based on single-lead ECG rather than clinical level 12-lead ECG, and thus only limited arrhythmia identification can be performed.

### SUMMARY

Some embodiments of the present application provide an ECG image processing method, device, medium, and an electrocardiograph.

In a first aspect, some embodiments of the present application provide an ECG image processing method, the method includes: receiving the ECG image; extracting a feature map of the ECG image, and reducing the feature map to obtain an attention map, wherein the attention map represents a specific section of the ECG image; extracting a feature matrix from the feature map and the attention map using a bilinear attention pooling, wherein the feature matrix includes a feature quantity corresponding to the specific section; obtaining an expression matrix using an adaptive weight learning and a weighted fusion of the feature matrix by a multi-headed self-attention processing, wherein the expression matrix represents an abnormal section in the ECG image; and classifying the ECG image with a plurality of labels based on the expression matrix, wherein the plurality of labels indicate an abnormal type corresponding to the abnormal section.

In a possible implementation of the first aspect, extracting the feature map of the ECG image includes: extracting the feature map of the ECG image based on an inception-v3 framework, wherein the feature map is denoted as F∈R^{H×W×M}, F represents the feature map, R represents the ECG image, H represents a height of the ECG image, W represents a width of the ECG image, and M represents a number of channels of the ECG image.

In a possible implementation of the first aspect, the feature map is calculated by a convolution operation with a kernel size of 1 to obtain the attention map, where a reduced dimension may be configured as any value from 1 to 32.

In a possible implementation of the first aspect, extracting the feature matrix from the feature map and the attention map using the bilinear attention pooling, wherein the feature matrix includes the feature quantity corresponding to the specific section, includes: splitting the attention map as shown in a following equation:

$$A = {⋃}_{i = 1}^{N} {a}_{i} ,$$

wherein A∈R^{H×W×N} represents the attention map, aᵢ∈R^{H×W} represents i-th part of the ECG image, and N represents a number of maps into which the attention map would be segmented; obtaining matrix pᵢ corresponding to the i-th part by multiplying each element of the attention map aᵢ with the feature map respectively, as:

pᵢ=g(aᵢ F)(k=1,2, . . . ,N),

wherein ⊙ represents elementary multiplication and g(.) represents pooling operation; and obtaining matrix p₁, p₂, . . . , p_{N} in turn by pooling for N times over M channels and combining the matrix p₁, p₂, . . . , p_{N} into an N×M feature matrix P∈R^{N×M} In a possible implementation of the first aspect, obtaining the expression matrix using the adaptive weight learning and the weighted fusion of the feature matrix by the multi-headed self-attention processing, wherein the expression matrix represents the abnormal section in the ECG image, comprises: using a formula to calculate an attention function as:

$$Attention ( Q , K , V ) = softmax ( \frac{Q {K}^{T}}{\sqrt{d}} ) V ,$$

wherein Q=V=K=P, Attention represents the attention function, softmax represents a mathematical function named as softmax transforming an input vector data into probability values between (0, 1), K^{T} represents a transpose of matrix K, and d represents a number of hidden units; focusing different parts of the value vector channel by using h heads, wherein for the i^{th} head, an output attention matrix is calculated as:

Headᵢ=Attention(QWᵢ^{Q},KWᵢ^{K},VWᵢ^{V}),

wherein Headᵢ represents an output vector of i^{th} self-attention mechanism, Wᵢ^{Q}∈R^{N×d/h}, Wᵢ^{K}∈R^{N×d/h}, Wᵢ^{V}∈R^{N×d/h} represent weights corresponding to vectors Q, K, and V of the output vector of the i^{th} self-attention mechanism, respectively; and summing h attention matrices and obtaining the expression matrix by a one-dimensionalization operation of a linear layer; wherein a multi-headed self-attention processing stitches together the output vectors of a plurality of self-attention mechanisms, and the expression matrix is obtained by the one-dimensionalization operation of the linear layer.

In a possible implementation of the first aspect, classifying the ECG image with the plurality of labels based on the expression matrix, comprises: generating a first label using a long and short-term memory model based on the expression matrix; and obtaining the plurality of labels by generating a second label based on the ECG image and the first label.

In a possible implementation of the first aspect, after receiving the ECG image, the ECG image is converted into a preset format and the ECG image is pre-processed with at least one of removing a background watermark, removing horizontal and vertical axis auxiliary lines, and performing precise segmentation of a lead data.

In a second aspect, some embodiments of the present application provide an electrocardiogram image processing device, including: a memory for storing an instruction executed by one or more processors of a system, and a processor, being one of the processors of the system, for executing the instruction to implement a possible implementation of the method of the first aspect.

In a third aspect, some embodiments of the present application provide a computer readable storage medium encoded using a computer program, wherein the computer readable storage medium has an instruction stored thereon, the instruction when executed on a computer causing the computer to perform a possible implementation of the method of the first aspect.

In a fourth aspect, some embodiments of the present application provide an electrocardiograph, including: a collecting device configured to collect body surface ECG information; and a processing device, communicatively connected to the collecting device, configured to receive the body surface ECG information, where the processing device includes a memory and a processor; wherein the memory is configured to store instruction executed by one or more processors of a system; and the processor is configured to form an ECG image based on the ECG information and execute a possible implementation of the method of the first aspect.

Compared to the prior art, the effect of the present application is in that by processing the ECG image in an end-to-end training manner, i.e. from image input to final formation of interpretation results, during which no human intervention is required. The present application achieves direct interpretation of the ECG image without being limited to the use of conventional digital signals, while being able to capture subtle differences in the ECG image for classifying ECG abnormalities with high noise immunity.

### BRIEF DESCRIPTION OF DRAWINGS

FIG. 1 illustrates a diagram of an application scenario for an ECG image processing method according to some embodiments of the present application.

FIG. 2 illustrates a network structure diagram for the ECG image processing method according to some embodiments of the present application.

FIG. 3 illustrates a block diagram of a hardware structure for the ECG image processing method according to some embodiments of the present application.

FIG. 4 illustrates a sample data in a training dataset according to some embodiments of the present application.

FIG. 5 illustrates a flowchart of the ECG image processing method according to some embodiments of the present application.

FIG. 6(a) illustrates an attentional thermal map generated by T-band variation image according to some embodiments of the present application.

FIG. 6(a) illustrates an attentional thermal map generated by ST-band variation image according to some embodiments of the present application.

FIG. 7 illustrates a schematic diagram of an ECG image processed by a bilinear attention pooling according to some embodiments of the present application.

FIG. 8 illustrates a schematic diagram of an ECG image processed by a multi-headed self-attention according to some embodiments of the present application.

### DETAILED DESCRIPTION OF EMBODIMENTS

Illustrative embodiments of the present application include, but are not limited to, an electrocardiogram image processing method, device, medium, and an electrocardiograph.

It may be understood that the ECG image processing method provided by the present application may be implemented on a variety of electronic devices, including, but not limited to, servers, distributed server clusters including multiple servers, cell phones, tablets, laptops, desktop computers, wearable devices, head-mounted displays, mobile email devices, portable game consoles, portable music players, reader devices, personal digital assistants, virtual reality or augmented reality devices, televisions with one or more processors embedded or coupled thereon, and other electronic devices.

It may be understood that the ECG image processing method provided by the present application may be directed to three types of ECG: static ECG, dynamic ECG and exercise ECG. When applied to longer duration dynamic ECG and exercise ECG, slices may be made for a specific length of time (e.g. 5 s, 10 s), and each slice may be used as an input in a form of an image.

It may be understood that in some embodiments of the present application, the processor may be a microprocessor, a digital signal processor, a microcontroller, etc., and/or any combination thereof. According to another aspect of the present application, the processor may be a single-core processor, a multi-core processor, etc., and/or any combination thereof.

The embodiments of the present application will be described in further detail below in combination with accompanying drawings.

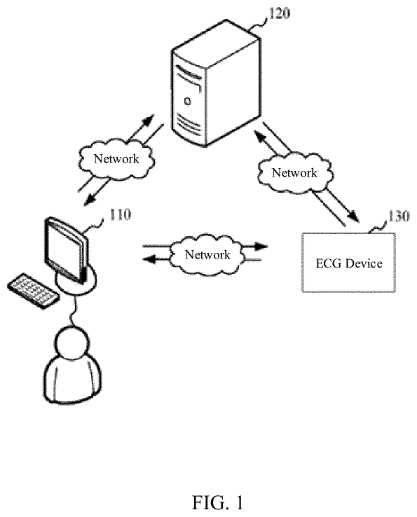

FIG. 1 illustrates an application scenario for an ECG image processing method according to some embodiments of the present application. Specifically, as shown in FIG. 1, the ECG image processing method is applied to an ECG image processing system. The ECG image processing system includes a terminal 110, a server 120, and an ECG device 130. The terminal 110, the server 120, and the ECG device 130 are connected to each other via a network. The network may include various connection types, such as wired communication links, wireless communication links, cloud or fiber optic cables, etc. Specific examples of the above network may include Internet provided by a communication provider of the terminal 110.

The terminal 110 may be a device installed with an end-to-end intelligent pre-screening triage system or computer-assisted clinical decision support system, specifically a desktop terminal or a mobile terminal. The mobile terminal specifically may be at least one of a cell phone, a tablet, a laptop, etc.

The server 120 may be implemented with a standalone server or a server cluster of multiple servers.

The ECG device 130 is a medical device that automatically records bioelectrical signals (ECG signals) generated by myocardial excitation during heart activity and forms ECG images. The ECG device 130 is commonly used for clinical diagnosis and scientific research. According to a principle of the image processing method provided by the method of the present application, the image processing method may also be used for an image output from other medical devices. So the ECG device 130 herein may be replaced with a CT (Computed Tomography) machine, a MRI (Magnetic Resonance Imaging) device and an ultrasound diagnostic instrument, a X-ray machine, an electrocardiogram equipment and an electroencephalogram equipment, etc.

ECG information is collected through electrodes at a body surface and transmitted to the ECG device 130 via lead wires. The ECG device 130 forms an ECG image using the collected ECG information from the body surface and transmits the ECG image to the server 120. The server 120 stores the ECG image and transmits the ECG image to the terminal 110. The terminal 110 receives the ECG image from the server 120, processes the ECG image using the electrocardiogram image processing method of the present application, and finally outputs an electrocardiogram image with a plurality of labels.

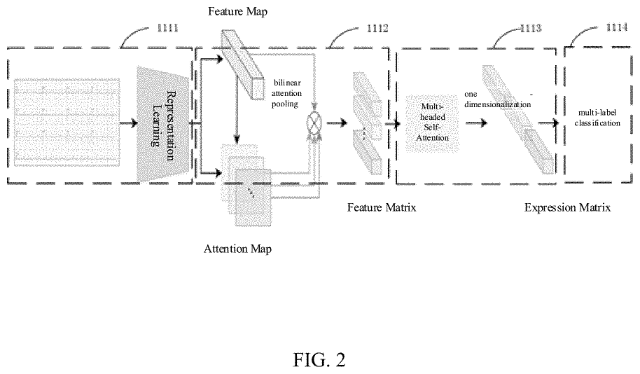

A technical solution of a network structure applied to the scenario shown in FIG. 1, corresponding to the ECG image processing method shown in FIG. 2 is described in detail, according to some embodiments of the present application. The method of the present application is directed to identify an extracted section from the ECG image and then perform adaptive multi-label classification according to the section's importance in an image abnormality classification in the medical field. As shown in FIG. 2, the network structure includes four modules, that is, a feature extraction module 1111, a feature matrix learning module 1112, an abnormal section learning module 1113 and a classification learning module 1114. The feature extraction module 1111 extracts a feature map by learning high-dimensional features from the image via representation learning, and obtains an attention map using attention mechanism based on fine-grained representation so as to represent the section in the ECG image. The feature matrix learning module 1112 learns from the section based on a weakly supervised bilinear attention pooling, in order to obtain a feature matrix representing a specific waveform. The specific section here is discriminated adaptively based on a multi-label corresponding to an input sample data set, which may be a specific wave, interval or band. The multi-label and the corresponding sample data set will be described in detail below. The abnormal section learning module 1113, based on a multi-headed self-attention processing, performs in-weight learning of the specific section and performs a one-dimensional linear operation on the obtained matrix to obtain an expression matrix representing an abnormal section in the ECG image. The classification learning module 1114, based on a long-short memory network, classifies the abnormal section with multiple labels to obtain an ECG image with multiple labels.

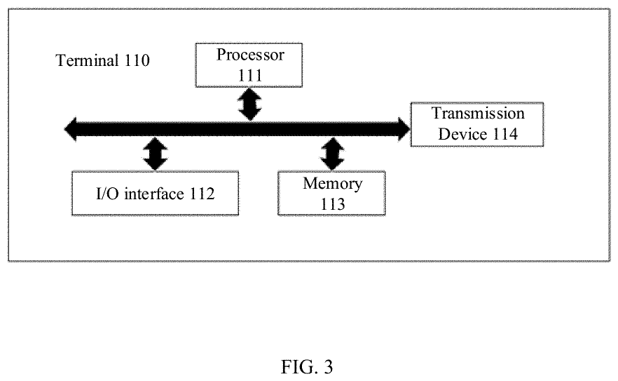

Some method implementations provided in the present application may be performed in the terminal 110. FIG. 3 illustrates a block diagram of a hardware architecture of the ECG image processing method according to some embodiments of the present application. As shown in FIG. 3, the terminal 110 may include one or more (only one is shown) processors 111, an input and output (I/O) interface 112 for interacting with a user, a memory 113 for storing data and a transmission device 114 for communication. The processors 111 may include, but are not limited to, a processing device such as a central processing unit CPU, an image processor GPU, a digital signal processor DSP, a microprocessor MCU, or a programmable logic device FPGA. It may be understood by those skilled in the art that the structure shown in FIG. 3 is only schematic and it does not limit the structure of the above electronic device. For example, the terminal 110 may also include more or fewer components than those shown in FIG. 3, or have a different configuration than that shown in FIG. 3.

The I/O interface 112 may be connected to one or more displays, touch screens, etc., for displaying data transmitted from the terminal 110. The I/O interface 112 may also be connected to a keyboard, stylus, trackpad, and/or mouse, etc., for inputting user commands such as, select, create, edit, etc.

The memory 113 may be used to store software programs and modules for databases, queues, application software, such as program instructions/modules corresponding to the ECG image processing method in some embodiments of the present application. The processor 111 performs various functional applications as well as data processing to implement the ECG image processing method described above, by running the software programs as well as modules stored in the memory 113. The memory 113 may include a high-speed random memory, and a non-volatile memory such as one or more magnetic storage devices, a flash memory, or other non-volatile solid state memory. In some examples, the memory 113 may further include a memory that is remotely located relative to the processor 111, which may be connected to the terminal 110 via the network. An example of the network includes, but is not limited to, Internet, a corporate intranet, a local area network, a mobile communication network, and combinations thereof.

The transmission device 114 is configured to receive ECG images uploaded via the ECG device 130 transmitted by the server 120 or to send a processed data to the server 120 via the network. The network may include various connection types such as wired, wireless communication links, cloud or fiber optic cables, etc. Specific examples of the network described above may include the Internet provided by the communication provider of the terminal 110.

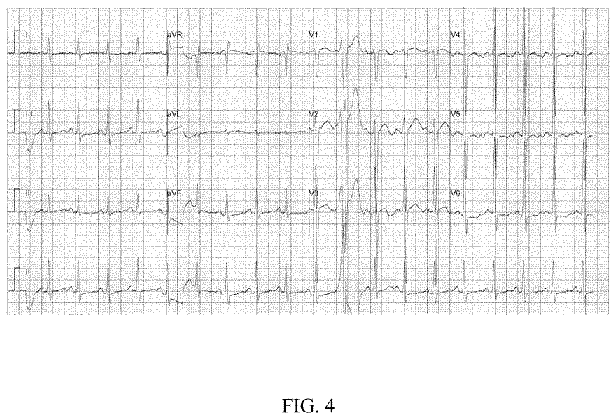

Before explaining the ECG image processing method according to the present application, the data set needs to be trained. FIG. 4 illustrates a sample data in the training data set according to some embodiments of the present application. Data from January 2015 to March 2015 from a primary hospital in China is collected to form an initial data set. The initial data set includes 27271 ECG images involving 27120 participants. The age distribution is 57.6±17.9 years, with 52.5% of females and 46.6% of males. Each recording is stored as a 12-lead ECG image in which four waveforms were presented in the ECG image. Each of the first three waveforms includes four lead signals with a 10-second duration and a 2.5-second duration per lead. The fourth waveform is a 10-second duration signal for lead II. Different leads provide different signal amplitudes and intervals. The 12-lead ECG image is the most widely used ECG image recording technique in clinical practice. The 12 leads include six anterior leads (V1, V2, V3, V4, V5, V6), three limb leads (I, II, iii), and three enhanced Limb leads (aVR, aVL, aVF). Each lead views the heart from a different angle. All images are stored in Portable Network Graphics (PNG) format. All of these ECG images are labeled for three independent classification tasks: noise, rhythm, and ST-segment abnormalities (ST). Table 1 below shows a detailed statistical result for each classification. Each ECG image recording is annotated by multiple clinical ECG image experts. The clinical ECG image experts use a web-based ECG image annotation tool, which is designed for the label. A majority voting strategy is used to ensure consistency of annotations between different experts.

TABLE 1

| | Number of | |
|---|---|---|
| Task | classifi- | |
| name | cation | Name of classification |
| Noise | 5 | Normal, mechanical wave interference, EMG |
| | | interference, baseline instability, baseline |
| | | drift |
| Rhythm | 16 | Normal, pacing ECG pacing, right-sided heart, sinus |
| | | arrest, sinus node wandering rhythm swing rhythm, |
| | | sinus bradycardia, sinus tachycardia, sinus |
| | | tachycardia, atrial tachycardia, junctional escape, |
| | | paroxysmal supraventricular tachycardia, atrial |
| | | flutter atrial flutter, atrial fibrillation atrial |
| | | fibrillation, ventricular fibrillation ventricular |
| | | fibrillation, nonparoxysmal connected tachycardia, |
| | | ventricular tachycardia |
| ST | 6 | Normal, T-wave change, ST-T segment change, |
| | | T-wave hyperacusis, Ptf V1 abnormal, J-point |
| | | elevated |

On basis of this dataset, data augmentation is used to generate a more robust training dataset. Image data augmentation is a technique that may be used to artificially scale the size of the training dataset by creating modified versions of the images to build a better deep learning model. It is worth noting that the enhanced data cannot be directly added to the training set in most prior ECG image processing because they are very sensitive to distortions in the temporal digital data, which can significantly degrade performances in the test set. However, since the input in the present application is a two-dimensional ECG image, overfitting can be effectively reduced and a balanced distribution between classes can be maintained by modifying the image with appropriate enhancements.

This advantage is particularly important in medical data analysis because most medical data are unbalanced and characterized by a large number of normal conditions and a very small number of abnormal conditions. With an increase of data, high specificity and sensitivity may be achieved in the present application. Due to the characteristics of ECG, the ECG data is increased by shifting in the present application. Shifting ECG images to left, right, up or down may avoid position shifts in the data. For example, if all abnormal sections in the training dataset are centered without shifting, it is difficult to process test samples with abnormal sections only at a corner. Thus the training images may be set to move randomly within 10% width in the horizontal direction and 5% height in the vertical direction.

Based on the ECG image training data set and the corresponding image label, some embodiments of the present application are directed to extract a distinguishable section from the ECG image, which indicate a potential abnormal section, and then perform adaptive weight learning based on the extracted distinguishable section to make the abnormal section more prominent, and then perform weighted fusion of features and one-dimensional processing, so as to perform multiple label classification. That is, based on a previous label and the ECG image, a next label is obtained based on the label classification.

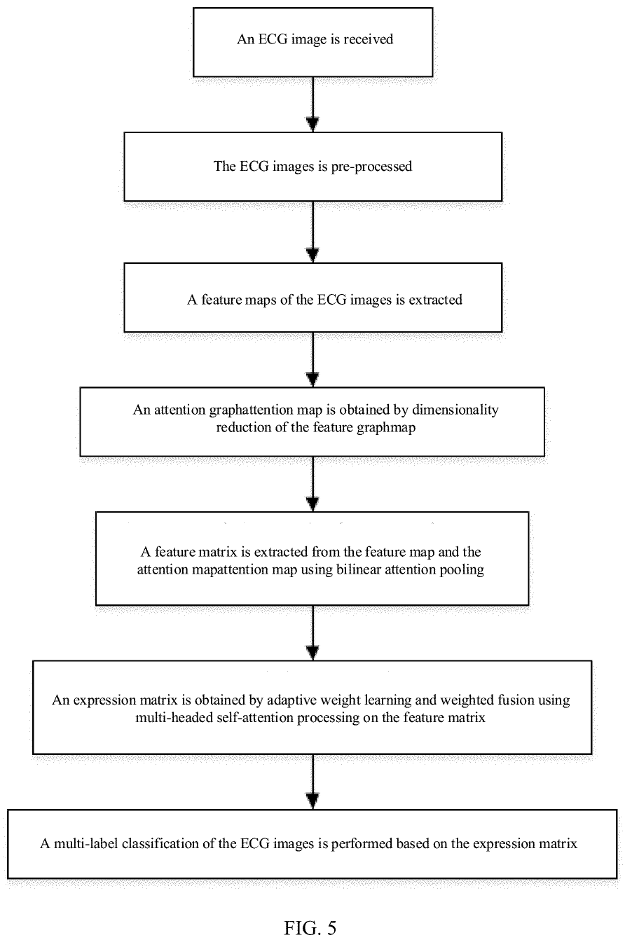

FIG. 5 illustrates a flowchart of the ECG image processing method according to some embodiments of the present application. As shown in FIG. 5, in some embodiments, the ECG image processing method may include the following steps.

In S1, an ECG image is received. The input ECG image is commonly in an image format of jpg, svg, png, pdf, etc. Both the format and size of the image may be set as required. For example, the ECG image may be uniformly converted into 150*300 png images as input.

In S2, the ECG image is pre-processed. For example, a background watermark may be removed, a horizontal axis auxiliary line and a vertical axis auxiliary line may be removed, and the lead data may be precisely segmented. For example, the information of all leads or some of the leads of interest may be extracted from a 12-lead image by image extraction and segmentation, so as to obtain a specific characterization of a plurality of individual leads.

In S3, a feature map of the ECG image is extracted. Compared with other convolutional neural networks, inception-v3 has higher accuracy and efficiency. Therefore, in order to be able to extract global variables that reflect the content of ECG images as completely as possible, inception-v3 is used as backbone framework in the present application. Firstly, based on the backbone framework inception-v3, features are aggregated from the input raw ECG image to learn the feature map of the ECG image by representation learning. The ECG image feature map is represented as F∈R^{H×W×M}, where F represents the feature map, R represents the ECG image, H represents the height dimension of the ECG image, W represents the width dimension of the ECG image dimension, M represents a number of channels of the ECG image. In this way, the input ECG image is represented by a set of feature maps, each of which represents a degree of matching to the ECG image on a given spatial pattern. This process can effectively increase the information of internal representation contained in the image.

In S4, an attention map is obtained by dimension reduction of the feature map. In order to obtain a fine-grained representation of a potentially discernible section, an attention mechanism is used to discover the importance of each feature map, i.e., to learn to identify a specific section from the feature map to form the attention map. The attention map is obtained by performing one or more convolutional layer operations on the feature map. Each element in the attention map represents a part of the ECG image, which may be a specific wave, interval, band, or a length of the wave. After a convolution operation with a kernel size of 1, the feature map is reduced to obtain the attention map. The reduced dimension may be configured as any value from 1 to 32, generally set to 32.

In S5, a feature matrix is extracted from the feature map and the attention map using bilinear attention pooling. For a class without any annotations, a set of partial representations needs to be obtained from the section. In order to filter out irrelevant or weakly relevant regions used to detect anomalies, such as a background and uninformative signal segments, an attention mechanism is introduced in the present application to apply the attention map through a series of convolution operations to identify a key section from the learned feature graph, i.e., to extract a fine-grained feature matrix from the section. The series of convolution operations is called Bilinear Attention Pooling (BAP). The feature matrix is extracted by combining features from two information sources. In the present application, the first information source is based on the output features of the backbone feature network, i.e., the feature map. The second information source is the attention map obtained from the feature map by one or more layers of convolution. The attention map is supervised to learn a feature distribution of the ECG image. The attention map is multiplied element by element with the feature map. Then, a bilinear attention pooling is applied in each section and the resulting feature matrix is flattened and cascaded. Different waveform parts are extracted from each row in the final feature matrix.

Specifically, the operation is as follows.

The described attention map is partitioned into N maps as shown in Equation (1):

$$A = {⋃}_{i = 1}^{N} {a}_{i} , \qquad ( 1 )$$

where A∈R^{H×W×N} represents the attention map, aᵢ∈R^{H×W} represents the i-th part of the ECG image, and N represents the number of maps into which the attention map will be segmented.

Each element of the attention map aᵢ is multiplied with the feature map separately to obtain matrix pᵢ corresponding to the i-th part as follows.

pᵢ=g(aᵢ_{_{⊙}}F)(k=1,2, . . . ,N), (2)

where ⊙ represents elemental multiplication and g(.) represents pooling operation.

Pooling is performed for N times over M channels to obtain matrix p1, p2, . . . , pN in turn. The matrix p1, p2, . . . , pN, are combined into an Nx M feature matrix P∈R^{N×M}.

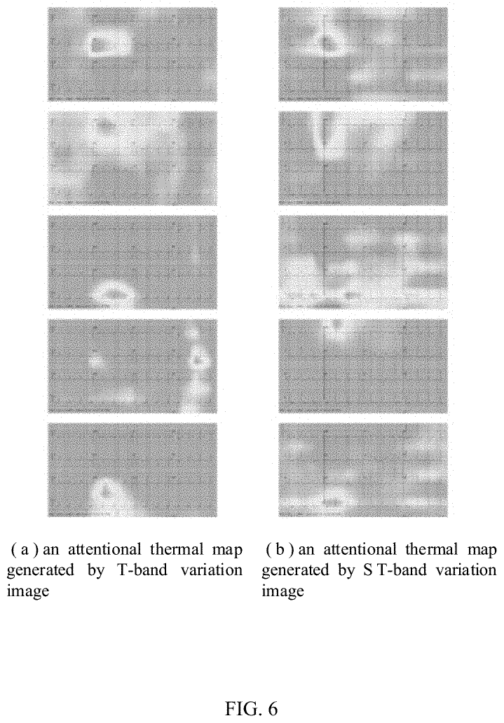

As a result, different features that represent specific waveform parts of each attention map are obtained. This process is completely unsupervised and has the advantage of being scalable to large-scale data. FIG. 6(a) illustrates an attentional thermal map generated by T-band variation image, according to some embodiments of the present application. FIG. 6(b) illustrates an attentional thermal map generated by ST-band variation image, according to some embodiments of the present application. In FIG. 6(a) and FIG. 6(b), attention maps obtained by automatic learning on the ECG image are shown, which helps to visualize the effectiveness of weakly supervised attention learning. According to the BAP operation, in the method a predetermined number of feature matrixes are extracted from an original image. For example, the number of feature matrixes found from the attention map is set to 32, and then the top 5 feature matrices with the highest attention are selected. Specifically, five sections are highlighted in FIG. 6(a), which shows abnormal T-wave changes in the ECG image. Most of these highlighted sections have the same x-coordinate but are located on different leads. This is because, when T-wave abnormalities occur, it is possible that they are captured by multiple leads at the same time, which is consistent with the ECG specialist's approach to the clinical phenomenon and review of this type of abnormality. Similarly, in FIG. 6(b), the section discovered for ST-band change detection is also present at the same time, indicating that the method can find a meaningful discriminative section.

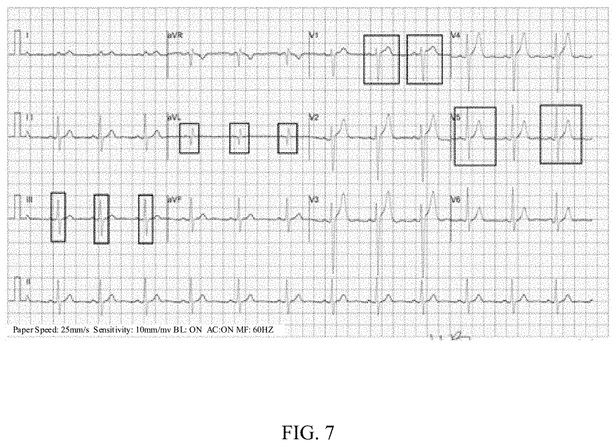

FIG. 7 illustrates a schematic diagram of an ECG image that has undergone a bilinear attention pooling according to some embodiments of the present application. When the number of feature matrices found from the attention map is set to 10, the ECG image is shown in FIG. 7, where each row of the feature matrix P represents a specific section, and where there are 10 rows of the feature matrix.

In S6, an expression matrix is obtained by adaptive weight learning and weighted fusion using multi-headed self-attention processing on the feature matrix. After obtaining the feature matrix, adaptive weighted fusion is performed to obtain more distinguishing features. Multi-head self-attention is an attention mechanism associated with different positions of a single sequence, with the aim of computing representations of the same sequence. Its effectiveness has been demonstrated in various applications, such as natural language understanding, abstract abstraction and image description generation. In contrast, the present application makes use of a multi-headed self-attention mechanism to better integrate information from multiple discovery sections. Unlike an original work that applied the mechanism to sequence-to-sequence generation tasks, the mechanism is applied in image classification tasks in the present application.

The multi-headed self-attention mechanism includes two main components: self-attention and multi-headed self-attention. The self-attention mechanism allows all sections to interact with each other and find out where they should pay more attention. The self-attention mechanism also outputs a summary of these interactions and attention scores. The multi-headed self-attention mechanism is a supervised learning and therefore also trained. The expression matrix is obtained by using the multi-headed self-attention mechanism to learn adaptive weights and fuse on the feature matrix. The expression matrix represents abnormal sections in the ECG image, including: at least one of specific waves, intervals or bands.

The attention function is calculated using equation (3).

$$Attention ( Q , K , V ) = softmax ( \frac{Q {K}^{T}}{\sqrt{d}} ) V , \qquad ( 3 )$$

where Q=V=K=P, Attention represents the attention function, softmax represents a mathematical function named softmax which transforms the input vector data into probability values between (0, 1), K^{T} represents a transpose of the matrix K, and d represents the number of hidden units.

In Equation (3), a scaled dot product attention layer is applied, and its output is a weighted sum of values, with the weight of each value determined by the dot product queried in all queues.

h heads are used to focus different sections of the value vector channel, for the i^{th} head, the output attention matrix is calculated as shown below.

Headᵢ=Attention(QWᵢ^{Q},KWᵢ^{K},VWᵢ^{V}), (4)

where Headᵢ represents an output vector of the i^{th} self-attention mechanism, Wᵢ^{Q}∈R^{N×d/h}, Wᵢ^{K}∈R^{N×d/h}, and Wᵢ^{V}∈R^{N×d/h} represent the weights corresponding to the vectors Q, K, and V of the output vector of the i^{th} self-attention mechanism, respectively.

The expression matrix is obtained by summing the h attention matrices and by the one dimensionalization operation of the linear layer. Where MultiHead stands for multi-headed self-attention processing, Concat stands for adding the matrices together, h stands for the number of output vectors of the self-attention mechanism, and W^{h} stands for the weight of the matrix multiplication operation with the summed matrix.

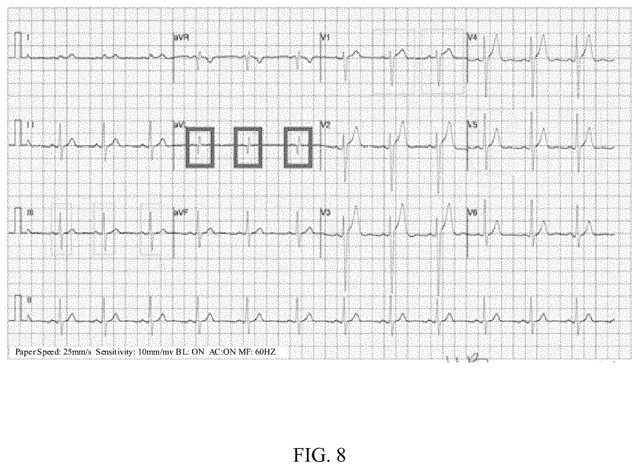

FIG. 8 shows a schematic diagram of an ECG image operated by a multi-headed self-attention mechanism according to some embodiments of the present application. As shown in FIG. 8, after the adaptive weight learning for each abnormal section is performed in the multi-headed self-attention mechanism, the section with higher weight indicates the higher importance of the abnormality, corresponding to the section with thicker border in FIG. 8. The section with lower weight indicates the lower importance of the abnormality, corresponding to the section with thinner border in FIG. 8.

The multi-headed self-attention processing stitches together the output vectors of multiple self-attention mechanisms, and after the one-dimensionalization operation of the linear layer, the expression matrix is obtained.

The multi-headed self-attention mechanism stitches the output vectors of multiple self-attention mechanisms together. After the one dimensionalization operation of the linear layer, the expression matrix is obtained. The multi-headed self-attention mechanism has many advantages over CNN. Firstly, unlike CNN, the multi-headed self-attention mechanism is not limited to a fixed window size, which means that it can be easier to fuse any learned sections regardless of the original positions of these features in the image. Secondly, the multi-headed self-attention mechanism uses an image feature to generate output vectors, which makes it easier to propagate gradients than convolutional operations.

In S7, a multi-label classification of the ECG image is performed based on the expression matrix. Based on a long and short-term memory model, iterative inference is performed to generate abnormal labels sequentially. That is, one abnormal label is generated each time. Based on the image and the previous label, the next label is obtained. For example, in the first time, a label of avl qr type may be obtained based on the image feature. In the second time, a label of sinus rhythm may be obtained based on the image feature and the label of avl qr type, until all possible labels are obtained. After a label of bradycardia is obtained, the label of tachycardia which is contrary to it may not be obtained, thus ensuring accuracy of the label. Conventionally, only one label can be obtained for one ECG image in the prior art, but in the technical solution of the present application, multiple labels may be obtained, thus significantly reducing rate of missed diagnosis as well as misdiagnosis.

To this end, extensive experimental validation is also performed. The proposed model and baseline are implemented in Python with TensorFlow. In addition, the experimental system in the present application includes two Intel Xeon E5 CPUs, 64 GB of main memory and two NVIDIA K20m GPUs. The corresponding software versions are TensorFlow r1.15, CUDA 10.0 and CUDNN 7.5.

First, attention map is set to be obtained by performing a 1×1 convolutional reduction of the feature map, with h set to 32. An Adam optimizer is used, where R31=0.9, 32=0.999, the batch size is set to 32. The initial learning rate is set to 0.001, followed by a decay factor of 0.1 after every 20 periods. In order to mitigate data imbalance, optimal bias initialization is applied to the last layer.

Considering that the method involves tasks of ECG image classification, fine learning and general image classification, the chosen baselines may be divided into three groups.

The first group includes a fine-grained image classification method, including.
1. A³M: For A³M, a global feature is learned by class classification, while a local feature is learned by attribute prediction. These two features are then refined into final features by an attribute class reciprocal attention module. Therefore, A³M requires additional attribute annotations.
2. B-CNN: B-CNN extracts the feature map from two independent CNN main chains and combines them via bilinear aggregation. Then the bilinear combination is normalized and used for the classification task.
3. WS-BAN: In order to extract distinguished local features by weakly supervised learning, WS-BAN learns attention mapping by attention regularization and attention deficit. Then a bilinear attention pooling is executed to extract sequential section features that are considered as the final feature representations of the classification task.
4. PC: Pairwise confusion (PC) regularization is introduced to make the predicted probability distributions closer and improve the generalization performance of the model.

The second group includes ECG image classification methods, including.
1. ECG image-CNN: It is a deep 2D CNN for ECG image arrhythmia classification, including 6 convolutional layers, 3 maximum pooling layers and 2 dense layers. Xavier initialization and exponential linear units (ELU) are used.
2. 34-layer CNN: The network contains 33 layers of convolution, followed by fully connected layers and Sofmax, and takes the time series of the original ECG image signal as input. All 1D convolutional layers may be replaced with 2D convolutional layers for comparison.

Finally, since the input is image classification, the present application is also compared with some widely used general image classification frameworks, including VGG16, Inception-v3, Resnet50 and Efficient Net-b0. Note that for a fair comparison, methods that use additional data and key-section annotations are not included. Unless otherwise stated, all baselines share the same main chain.

In all experiments and evaluations, 5-fold cross-validation, with case separation in horizontal direction of the image, is used. Overall accuracy, recall and F1 are used to evaluate the classification performance. In addition, the confusion matrix is calculated and the reservation and recall for each specific class are measured. All these measurements are given in the following equation.

$$Precision = \frac{T P}{T P + F P} , \qquad ( 5 )$$

$$Recall = \frac{T P}{T P + F N} , \qquad ( 6 )$$

$${F}_{1} = 2 \times \frac{Precision \times Recall}{Precision + Recall} , \qquad ( 7 )$$

where Precision indicates precision, Recall indicates recall, TP indicates true positive, FP indicates false positive, TN indicates true negative, and FN indicates false negative.

The proposed method was compared with the baseline on the above three abnormality detection tasks in the collected ECG image data. The results are shown in Table 2, Table 3 and Table 4, respectively. For each table, the within-class measurement (left of the table) and overall measurement (right of the table) with precision, recall, and F1 scores are listed separately. Due to space constraints, only the first 3 classes that includes most instances are listed.

TABLE 2
Comparison with baseline for classification of rhythm abnormality types.

| | Normal | Right Ventricular | Sinus Rythm | | Overall | |
|---|---|---|---|---|---|---|
| Method | Accuracy/Recall | Accuracy/Recall | Arrest/Recall | Overall | Recall | F1 |
| A³M | 0.964/0.966 | 0.815/0.852 | 0.880/0.918 | 0.931 | 0.930 | 0.930 |
| B-CNN | 0.952/0.951 | 0.805/0.856 | 0.891/0.924 | 0.925 | 0.932 | 0.927 |
| WS-BAN | 0.963/0.966 | 0.790/0.838 | 0.898/0.913 | 0.931 | 0.932 | 0.931 |
| PC | 0.948/0.965 | 0.786/0.818 | 0.883/0.887 | 0.909 | 0.915 | 0.910 |
| ECG-CNN | 0.945/0.972 | 0.769/0.831 | 0.893/0.887 | 0.897 | 0.921 | 0.909 |
| 34-layer CNN | 0.951/0.968 | 0.791/0.855 | 0.885/0.892 | 0.907 | 0.920 | 0.911 |
| VGG16 | 0.943/0.968 | 0.758/0.834 | 0.882/0.877 | 0.879 | 0.910 | 0.893 |
| Inception - v3 | 0.962/0.968 | 0.806/0.845 | 0.878/0.917 | 0.927 | 0.931 | 0.928 |
| Resnet50 | 0.968/0.963 | 0.792/0.852 | 0.884/0.917 | 0.932 | 0.933 | 0.932 |
| EfficientNet - b0 | 0.964/0.965 | 0.805/0.851 | 0.889/0.922 | 0.932 | 0.933 | 0.932 |
| Proposed | 0.970/0.964 | 0.827/0.858 | 0.922/0.926 | 0.935 | 0.934 | 0.934 |
| Method | | | | | | |

TABLE 3
Comparison with baseline for classification of noise types.

| | | | Mechanical | | | |
|---|---|---|---|---|---|---|
| | Normal | EMG | Interference | Overall | Overall | |
| Method | Accuracy/Recall | Accuracy/Recall | Accuracy/Recall | Accuracy | Recall | F1 |
| A³M | 0.948/0.980 | 0.770/0.578 | 0.500/0.147 | 0.927 | 0.935 | 0.928 |
| B-CNN | 0.953/0.964 | 0.719/0.724 | 0.500/0.013 | 0.931 | 0.933 | 0.932 |
| WS-BAN | 0.944/0.964 | 0.765/0.623 | 0.750/0.040 | 0.926 | 0.931 | 0.927 |
| PC | 0.950/0.954 | 0.734/0.676 | 0.514/0.253 | 0.931 | 0.936 | 0.932 |
| ECG-CNN | 0.936/0.980 | 0.753/0.446 | 0.500/0.040 | 0.914 | 0.926 | 0.913 |
| 34-layer CNN | 0.926/0.966 | 0.731/0.726 | 0.514/0.147 | 0.883 | 0.913 | 0.895 |
| VGG16 | 0.922/0.965 | 0.748/0.424 | 0.750/0.040 | 0.889 | 0.918 | 0.902 |
| Inception - v3 | 0.946/0.965 | 0.752/0.544 | 0.545/0.185 | 0.920 | 0.926 | 0.924 |
| Resnet50 | 0.944/0.952 | 0.739/0.572 | 0.528/0.046 | 0.919 | 0.921 | 0.919 |
| EfficientNet - b0 | 0.939/0.977 | 0.758/0.634 | 0.586/0.145 | 0.919 | 0.923 | 0.920 |
| Proposed | 0.953/0.981 | 0.772/0.729 | 0.500/0.253 | 0.936 | 0.942 | 0.938 |
| Method | | | | | | |

TABLE 4
Comparison with the baseline method used to classify ST anomaly types.

| | | T-band | ST-T | | | |
|---|---|---|---|---|---|---|
| | Normal | Alternation | Alternation | Overall | Overall | |
| Method | Accuracy/Recall | Accuracy/Recall | Accuracy/Recall | Accuracy | Recall | F1 |
| A³M | 0.905/0.964 | 0.668/0.601 | 0.546/0.160 | 0.863 | 0.876 | 0.865 |
| B-CNN | 0.912/0.948 | 0.660/0.622 | 0.553/0.253 | 0.860 | 0.871 | 0.862 |
| WS-BAN | 0.917/0.945 | 0.659/0.623 | 0.467/0.542 | 0.870 | 0.870 | 0.870 |
| PC | 0.910/0.950 | 0.653/0.585 | 0.547/0.333 | 0.859 | 0.870 | 0.861 |
| ECG-CNN | 0.908/0.947 | 0.659/0.630 | 0.508/0.280 | 0.857 | 0.872 | 0.861 |
| 34-layer CNN | 0.911/0.932 | 0.673/0.632 | 0.363/0.289 | 0.852 | 0.885 | 0.859 |
| VGG16 | 0.893/0.961 | 0.633/0.569 | 0.553/0.253 | 0.836 | 0.865 | 0.850 |
| Inception - v3 | 0.898/0.961 | 0.671/0.553 | 0.513/0.164 | 0.846 | 0.867 | 0.851 |
| Resnet50 | 0.908/0.966 | 0.634/0.569 | 0.552/0.048 | 0.843 | 0.862 | 0.845 |
| EfficientNet - b0 | 0.901/0.956 | 0.664/0.556 | 0.485/0.217 | 0.846 | 0.866 | 0.853 |
| Proposed | 0.931/0.937 | 0.675/0.659 | 0.557/0.475 | 0.876 | 0.877 | 0.876 |
| Method | | | | | | |

As can be seen in Tables 2-4, the proposed method in the present application outperforms all baseline methods for all overall measurements and within-class measurement. For all tasks, it improves the baseline results by about 3% in terms of accuracy, recall, and F1 score. Furthermore, the numerical-based ECG image classification methods: ECG image-CNN and 34-layer CNN do not achieve satisfactory performance in all tasks. Another interesting finding is that the average performance of the fine-grained classification group outperforms the normal image classification group in two of the three tasks, except for the rhythm abnormality detection task. The main reason for this is that in the tasks of noise and ST anomaly classification, the critical areas are usually relatively small and subtle, which are easier to be detect by a fine-grained based approach. But rhythm anomaly detection is more about measuring the overall frequency of the waveform rather than finding local discriminative sections, and thus the advantages of the fine-grain based method cannot be realized. Even so, because the fine-grained approach in the present application has a spatial attention mechanism on the learned section, this spatial attention mechanism is still able to obtain the whole frequency information by assigning a series of more precise height attention to successive sections, thus making the method in the present application still have a better overall robustness.

A second implementation of the present application relates to an electrocardiogram image processing device, including:
a memory for storing instructions executed by one or more processors of a system, and
a processor, being one of the processors of the system, for executing the instructions to implement any of the possible methods of the first aspect described above.

The first implementation is a method implementation corresponding to the present implementation, and the present implementation can be implemented with the first implementation in conjunction with each other. The relevant technical details mentioned in the first implementation are still valid in the present implementation, and will not be repeated here in order to reduce repetition. Accordingly, the relevant technical details mentioned in the present implementation may also be applied in the first implementation.

A third implementation of the present application relates to a computer storage medium using a computer program encoded with instructions stored on the computer readable medium which, when executed on the computer, enables the computer to perform any of the possible methods of the first aspect described above.

The first implementation is a method implementation corresponding to the present implementation, and the present implementation can be implemented with the first implementation in conjunction with each other. The relevant technical details mentioned in the first implementation are still valid in the present implementation, and will not be repeated here in order to reduce repetition. Accordingly, the relevant technical details mentioned in the present implementation may also be applied in the first implementation.

A fourth implementation of the present application relates to an electrocardiograph, including:
a collecting devices configured to collect body surface ECG information;
a processing device, communicatively connected to the collecting device, configured to receive the body surface ECG information, where the processing device includes a memory and a processor;
the memory configured to store instruction executed by one or more processors of a system;
the processor, configured to form an ECG image based on the ECG information and execute the instructions on the ECG image to implement any one of the possible methods of the first aspect described above.

The first implementation is a method implementation corresponding to the present implementation, and the present implementation can be implemented with the first implementation in conjunction with each other. The relevant technical details mentioned in the first implementation are still valid in the present implementation, and will not be repeated here in order to reduce repetition. Accordingly, the relevant technical details mentioned in the present implementation can also be applied in the first implementation.

It should be noted that each method implementation of the present application may be implemented in software, hardware, firmware, etc. Regardless of whether the application is implemented in software, hardware, or firmware, the instruction code may be stored in any type of computer-accessible memory (e.g., permanent or modifiable, volatile or non-volatile, solid-state or non-solid-state, fixed or media-replaceable, etc.). Similarly, the memory may be, for example, Programmable Array Logic (PAL), Random Access Memory (RAM), Programmable Read Only Memory (PROM), Read-Only Memory (ROM), Electrically Erasable Programmable ROM (EEPROM), disks, CD-ROMs, Digital Versatile Disc (DVD), etc.

It should be noted that each unit/module mentioned in each device implementation of the present application is a logical unit/module. A logical unit may be a physical unit, or a part of a physical unit, or may be implemented as a combination of multiple physical units. The physical implementation of these logical units is not the most important, while the combination of functions implemented by these logical units is the key to solving the technical problems presented in the present application. In addition, in order to highlight the innovative parts of the present application, the above-mentioned the device implementations of the present application do not introduce units that are less closely related to solving the technical problems presented in the present application, which does not indicate that there are no other units in the above-mentioned the device implementations.

It should be noted that in the claims and specification of this present application, relationship terms such as first and second are used only to distinguish one entity or operation from another, and do not necessarily require or imply any such actual relationship or sequence between those entities or operations. Further, the terms “includes,” “comprises,” or any other variation thereof are intended to cover non-exclusive inclusion, such that a process, method, article, or device that includes a set of elements includes not only those elements, but also other elements not expressly listed, or that also includes an element that is intended to be used for the purpose of the process, method, article, or device. Without further limitation, an element specified by the statement “includes a” does not preclude the existence of additional identical elements in the process, method, article, or device that includes the element.

Although the present application has been illustrated and described by reference to certain preferred embodiments of the present application, it should be understood by those of ordinary skill in the art that various modifications may be made thereto in form and detail without departing from the spirit and scope of the present application.

1. A method for processing an electrocardiogram (ECG) image, comprising:
receiving the ECG image;
extracting a feature map of the ECG image, and reducing the feature map to obtain an attention map, wherein the attention map represents a specific section of the ECG image;
extracting a feature matrix from the feature map and the attention map using a bilinear attention pooling, wherein the feature matrix includes a feature quantity corresponding to the specific section;
obtaining an expression matrix using an adaptive weight learning and a weighted fusion of the feature matrix by a multi-headed self-attention processing, wherein the expression matrix represents an abnormal section in the ECG image; and
classifying the ECG image with a plurality of labels based on the expression matrix,
wherein the plurality of labels indicate an abnormal type corresponding to the abnormal section.

2. The method according to claim 1, wherein extracting the feature map of the ECG image comprises:
extracting the feature map of the ECG image based on an inception-v3 framework, wherein the feature map is denoted as F∈R^{H×W×M}, F represents the feature map, R represents the ECG image, H represents a height of the ECG image, W represents a width of the ECG image, and M represents a number of channels of the ECG image.

3. The method according to claim 2, wherein reducing the feature map to obtain the attention map, wherein the attention map represents the specific section of the ECG image, comprises:
reducing the feature map by a convolution operation with a kernel size of 1 to obtain the attention map, wherein a reduced dimension may be configured as any value from 1 to 32.

4. The method according to claim 3, wherein extracting the feature matrix from the feature map and the attention map using the bilinear attention pooling, wherein the feature matrix includes the feature quantity corresponding to the specific section, comprises:
splitting the attention map as shown in a following equation:
$$A = {⋃}_{i = 1}^{N} {a}_{i} ,$$
wherein A∈R^{H×W×N} represents the attention map, aᵢ∈R^{H×W} represents i-th part of the ECG image, and N represents a number of maps into which the attention map would be segmented;
obtaining matrix pᵢ corresponding to the i-th part by multiplying each element of the attention map aᵢ with the feature map respectively, as:
pᵢ=g(aᵢF)(k=1,2, . . . ,N),
wherein ⊙ represents elementary multiplication and g(.) represents pooling operation; and
obtaining matrix p₁, p₂, . . . , p_{N} in turn by pooling for N times over M channels and combining the matrix p₁, p₂, . . . , p_{N} into an N×M feature matrix P∈R^{N×M}.

5. The method according to claim 4, obtaining the expression matrix using the adaptive weight learning and the weighted fusion of the feature matrix by the multi-headed self-attention processing, wherein the expression matrix represents the abnormal section in the ECG image, comprises:
using a formula to calculate an attention function as:
$$Attention ( Q , K , V ) = softmax ( \frac{Q {K}^{T}}{\sqrt{d}} ) V ,$$
wherein Q=V=K=P, Attention represents the attention function, softmax represents a mathematical function named as softmax transforming an input vector data into probability values between (0, 1), K^{T} represents a transpose of matrix K, and d represents a number of hidden units;
focusing different parts of the value vector channel by using h heads, wherein for the i^{th} head, an output attention matrix is calculated as:
Headᵢ=Attention(QWᵢ^{Q},KWᵢ^{K},VWᵢ^{V}),
wherein Headᵢ represents an output vector of i^{th} self-attention mechanism, Wᵢ^{Q}∈R^{N×d/h}, Wᵢ^{K}∈R^{N×d/h}, Wᵢ^{V}∈R^{N×d/h} represent weights corresponding to vectors Q, K, and V of the output vector of the i^{th} self-attention mechanism, respectively; and
summing h attention matrices and obtaining the expression matrix by a one-dimensionalization operation of a linear layer,
wherein a multi-headed self-attention processing stitches together the output vectors of a plurality of self-attention mechanisms, and the expression matrix is obtained by the one-dimensionalization operation of the linear layer.

6. The method according to claim 5, wherein classifying the ECG image with the plurality of labels based on the expression matrix, comprises:
generating a first label using a long and short-term memory model based on the expression matrix; and
obtaining the plurality of labels by generating a second label based on the ECG image and the first label.

7. The method according to claim 6, wherein after receiving the ECG image, the ECG image is converted into a preset format and the ECG image is pre-processed with at least one of removing a background watermark, removing horizontal and vertical axis auxiliary lines, and performing precise segmentation of a lead data.

8. An electrocardiogram image processing device, comprising:
a memory for storing an instruction executed by one or more processors of a system; and
a processor, being one of the processors of the system, for executing the instruction to implement the method for processing the ECG image of claim 1.

9. A computer readable storage medium encoded using a computer program, wherein the computer readable storage medium has an instruction stored thereon, the instruction when executed on a computer causing the computer to perform the method for processing the ECG image of claim 1.

10. An electrocardiograph, comprising:
a collecting device configured to collect body surface ECG information; and
a processing device, communicatively connected to the collecting device, configured to receive the body surface ECG information, where the processing device includes a memory and a processor,
wherein the memory is configured to store instruction executed by one or more processors of a system, and
wherein the processor is configured to form an ECG image based on the ECG information and execute the method for processing the ECG image of claim 1 on the ECG image.
